(19) United States
(12) Patent Application Publication
(10) Pub. No.: US 2007/0107074 A1
(43) Pub. Date: May 10, 2007

(54) Porcine Nuclear Transfer

(75) Inventors: Paul John Verma, Greenacres (AU); Mark Brenton Nottle, Bibaringa (AU)

Correspondence Address: FISH & RICHARDSON P.C., P.O. BOX 1022, MINNEAPOLIS, MN, 55440-1022, US

(73) Assignee: Garelag Pty Ltd., Victoria (AU)

(21) Appl. No.: 11/562,852

(22) Filed: Nov. 22, 2006

Related U.S. Application Data
(63) Continuation of application No. 09/646,479, filed on Jul. 28, 2004, now abandoned.

(30) Foreign Application Priority Data
Mar. 16, 1998 (AU) PP 2364
Dec. 15, 1998 (AU) PP 7720

(51) Int. Cl. A01K 67/027; C12N 15/87

(52) U.S. Cl. 800/17; 435/459

(57) ABSTRACT

A process for the production of nuclear transferred porcine embryonic cells which includes providing a porcine oocyte at the Metaphase II stage of development from which the nucleus is removed, transferring a porcine karyoplast at the G0 or G1 state into the oocyte to give a nuclear transferred porcine embryonic cell and optionally culturing the nuclear transferred cell in vitro to allow one or more cell divisions to give a plurality of nuclear transferred porcine embryonic cells is disclosed. Also disclosed is a process for the clonal generation or propagation of pigs which process includes providing a procine oocyte at the Metaphase II stage of development from which the nucleus is removed, transferring a porcine donor karyoplast at the G0 or G1 state into the oocyte to give a nuclear transferred porcine embryonic cell, and thereafter culturing the nuclear transferred cell in vitro to allow one or more cell divisions to give a plurality of nuclear transferred porcine embryonic cells, and thereafter transferring a plurality of porcine embryonic cells so produced into a pregnancy competent uterus of a female pig which at conclusion of the pregnancy term gives rise to one or more genetically identical off-spring.

This invention relates to porcine nuclear transfer processes for the production of nuclear transferred porcine embryonic cells, processes for the clonal generation of pigs, production of transgenic and genetically modified pigs, and pigs so produced.

The reconstruction of animal embryos by the transfer of a nucleus from a donor cell to either an enucleated oocyte or one cell zygote allows in theory the cloning of animals, that is, the production of genetically identical individuals. Practice is quite different. Whilst claims have been made that certain procedures have application across a wide range of animals, experience has shown that techniques which may be effective in the cloning of animals of one species either do not work in other species, give rise to embryos with a very low efficiency such that cloning would be impractical, or give rise to embryos which fail to develop on introduction to a pregnancy competent uterine environment of a recipient animal. For example, see Prather et al, (1989), Biology of Reproduction 41, 414-448.

WO 97/07668 and WO 97/07669 describe a nuclear transfer method involving donor cells resulting from serum starvation. The techniques of these applications fail to develop embryos capable of developing in a pregnancy competent uterine environment in many animals, and as a consequence are generally ineffective for cloned embryo production, and development, such as in pigs.

The present invention provides processes for the high efficiency production of nuclear transferred porcine embryonic cells capable of high efficiency development in the pregnancy competent porcine uterine environment to give clonal infant animals.

In accordance with one aspect of the present invention there is provided a process for the production of nuclear transferred porcine embryonic cells which includes providing a porcine oocyte at the Metaphase II stage of development from which the chromosomal material is removed, transferring a porcine karyoplast at the G0 or G1 (G0/G1) state into the oocyte to give a nuclear transferred porcine embryonic cell, and optionally culturing the cell in vitro to allow one or more cell divisions to give a plurality of nuclear transferred embryonic cells.

The nuclear transferred porcine embryonic cell may be incubated to-form a 2 to 32 cell stage or mass, such as a 2 to 16 cell mass (that is, a plurality of cells), whereafter the cell mass may be synchronized at the G0/G1 state. A nuclear transferred karyoplast may be isolated from the cell mass, and transferred into a second enucleated oocyte at the Metaphase II stage of development or to an enucleated zygote or later stage embryo or embryonic cell to give a second nuclear transferred cell, which may be cultured in vitro, to allow one or more cell divisions to give a plurality of nuclear transferred porcine embryonic cells.

Karyoplasts may be synchronized at the G1/S boundary state by use of DNA synthesis inhibitor which arrests the karyoplast at the G1 phase and/or use of a microtubule inhibitor which following removal of the microtubule inhibitor results in synchronization of said karyoplast at the G1 phase, and/or use of means which do not involve serum starvation of cells. Karyoplasts may be synchronized at the G0 phase by nutrient deprivation and/or chemical treatment.

In another aspect this invention relates to a process for the clonal generation or propagation of pigs which process includes providing a porcine oocyte at the Metaphase II stage of development from which the nucleus is removed, transferring a porcine donor karyoplast at the G0/G1 state into the oocyte to give an nuclear transferred cell, culturing the nuclear transferred cell in vitro to allow one or more cell divisions to give a plurality of nuclear transferred porcine embryonic cells, and thereafter transferring a plurality of porcine embryonic cells so produced into a pregnancy competent uterus of a female pig which at conclusion of the pregnancy term gives rise to one or more genetically identical off-spring.

A further aspect of this invention provides porcine embryonic cells and pigs when prepared according to the above process.

In another aspect of this invention there is provided a process as described above wherein the porcine karyoplast at the G0/G1 state is fused and activated in an enucleated porcine oocyte at the Metaphase II stage of development by application of one or multiple electrical pulses spaced in their order of application, or by other means of generating multiple transient increases in intracellular Ca levels.

In another aspect of this invention there is provided a cloned pig produced from a nuclear transferred porcine embryonic cell.

In another aspect of this invention there is provided use of cloned pigs in agriculture, for organ production, or oocyte and embryo production.

In one aspect of this invention there is provided a process for the production of nuclear transferred porcine embryonic cells. A porcine oocyte from which the nucleus is removed is fused with the nucleus of a porcine donor karyoplast A karyoplast is a donor nucleus, or the nucleus of a donor cell surrounded by an envelope of cytoplasm, or donor cell. Porcine oocytes at the Metaphase II stage of development may be readily collected from the oviducts of ovulating pigs. Ovulation may be induced by administering gonadotrophins of various species origin to the pigs. In the practice of the present invention, oocytes can be collected on appearance of the first polar body or as soon as possible after ovulation. Alternatively immature oocytes collected from the ovaries of living or slaughtered pigs may be matured in vitro to the Metaphase II stage which is readily observable by microscopic evaluation.

The nucleus is removed from the porcine oocyte at the Metaphase II stage by standard techniques, such as aspiration of the first polar body and neighbouring cytoplasm containing the metaphase chromosomes (see for example Smith & Wilmut (1989) Biol. Reprod. 40, 1027-1035), ultraviolet radiation (see for example Tsunoda et al (1988) J. Reprod. Fertil. 82, 173) or another enucleating influence.

The porcine karyoplast is transferred into the porcine oocyte at the Metaphase I] state as mentioned above. The karyoplast which is at the G1 or G0 state as will be described hereinafter, is transferred into the enucleated porcine oocyte by standard techniques in the field, such as cell fusion of the enucleated porcine oocyte and the karyoplasts (that is, as mentioned above, a cell or nucleus of a cell surrounded by an envelope of cytoplasm) or by direct injection of the karyoplast into the enucleated porcine oocyte. Established methods for inducing cell fusion include exposure of cells to fusion-promoting chemicals, such as polyethylene glycol (see, for example, Kanka et al, (1991), Mol. Reprod. Dev., 29, 110-116), the use of inactivated virus, such as sendi virus (see, for example, Graham et al, (1969), Wistar Inst. Symp. Monogr., 9, 19), and the use of electrical stimulation (see, for example, Willasden, (1986), Nature, 320, (6), 63-36 and Prather et al, (1987), Biol. Reprod., 37, 859-866). Use of electrical stimulation or cell fusion is preferred but by no means essential to this invention. By way of example, fusion of an enucleated oocyte with a donor cell may be accomplished by electro-pulsing in 0.3 M mannitol or 2.7 M sucrose solution. It has been surprisingly found by the inventors that activation by multiple electrical pulses spaced in their order of application gives rise to embryos capable of implantation and development to term unexpectedly superior to other methods. The same initial electrical pulse can be used to fuse the karyoplast and enucleated oocyte (simultaneous fusion and activation), or alternatively fusion and activation can be conducted sequentially when fusion occurs in Calcium-free medium. Activation by multiple electrical pulses results in multiple increases in intracellular calcium, mimicking the multiple transient increases that occur immediately following fertilisation. Multiple increases in intracellular calcium can also be achieved by other means, including by multiple treatments with chemical inducers such as the calcium ionophore ionomycin. These transient increases in intracellular calcium signal the resumption of meiosis. By way of example 2 to 6 electrical pulses may be delivered to the entities at an interval between each pulse of from one minute to sixty minutes, such as 2 pulses 30 minutes apart. Each pulse may be in the form of a set of pulses, such as 2 to 4 pulses, spread from each other by I to 20 seconds. DC pulses are generally used at a voltage such as 150 v/mm for a duration such as 60 μs, and generally with a pre- and post-pulse alternating current.

Direct micro injection of the karyoplast into an enucleated porcine oocyte may be carried out by conventional method, such as disclosed by Ritchie & Campbell, J. Reproduction and Fertility Abstract Series No. 15, page 60. As another example, a karyoplast may be introduced by injection into an enucleated porcine oocyte in a calcium free medium.

Enucleation of the porcine oocyte and transfer of the porcine donor karyoplast may be carried out as soon as the oocyte reaches the Metaphase II stage. This would generally coincide with the post-onset of maturation in vitro, after collection of ovaries from slaughtered ovulating pigs, or following hormone treatment in vivo.

The donor karyoplast, whether transferred directly into the cell, or transferred via fusion of the donor cell with the enucleated porcine oocyte is synchronized at the G1 or G0 state. In this regard, the cell cycle has four distinct phases, G1, S, G2 and M, as is well known in the art. G0 is a quiescent stage of low metabolic activity. The beginning event in the cell cycle is called start which takes place at the beginning of the G1 phase. Once a cell has passed through start, it passes through the remainder of the G1 phase, which is the pre-DNA synthesis stage. The second stage, the S phase, is the stage where DNA synthesis takes place. The G2 phase follows, which is the period between DNA synthesis and mitosis. Metaphase occurs during mitosis, which is referred to as the M phase.

Preferably, karyoplasts may be synchronized at the G1 state using a DNA synthesis inhibitor and/or use of a microtubule inhibitor which, on following removal of the inhibitor(s), results in synchronization of the karyoplast at the G1 state, or by means other than DNA inhibition, excluding serum starvation, for example cdk kinase inhibitors such as Butyrolactone I (Motlik et al (1998) Theriogeneology 49: 461-469). Examples of DNA synthesis inhibitors include: aphidicolin, hydroxyurea, cytosine arabinoside, 5-fluorouracil, n-ethylmalemide and etoposide. Any microtubule inhibitor may be used in this invention including nocodazole, colchecine or colcemid. Alternatively, a microtubule stabilizer such as, for example, taxol may be used. Karyoplasts may, for example, be synchronized at G1 by the use of a microtubule inhibitor such as nocodazole (to give a population of nuclei at the metaphase) followed by treatment with a DNA synthesis inhibitor such as aphidicolin in which the nuclei progress to an arrest at the G1 state. Alternatively only one of the aforementioned inhibitors may be utilised, or another means as discussed above which does not involve DNA synthesis inhibition. Karyoplasts may be synchronized in the Go state by nutrient deprivation, such as incubation in a low serum containing medium, as is known in the art, or by chemical treatment.

Donor karyoplasts (such as cells) may be incubated in a standard culture medium with a DNA synthesis inhibitor and/or microtubule inhibitor for a time sufficient to synchronize the cells at the G1 state. This can be readily observed by microscopic observations. DNA synthesis inhibitors and/or microtubule inhibitors may be used, for example, in an amount of from about 0.01 μg/ml to about 50 μg/ml, such as about 1-5 μg/ml culture medium. Microtubule inhibitors fix the cells at the M phase. After removal of microtubule inhibitor from the cell media, which can conveniently be done by washing the cells, cells pass to the G1 phase after about 30 minutes to 6 hours in a uniform manner such that a plurality of cells in the G1 phase can be conveniently prepared. A DNA synthesis inhibitor synchronises cells at the G1 phase. Removal of a DNA synthesis inhibitor from cell media allows the cell cycle to proceed. Similarly donor karyoplasts may be synchronized in the G0 state as described above.

Donor cells may be any porcine somatic cell, for example a foetal embryonic fibroblast cell, mammary cell, smooth muscle cell etc. Any somatic cell may be utilised. Porcine embryonic foetal fibroblast cells are particularly preferred. The donor cell may, by way of further example, be a porcine embryonic cell, such as a totipotent blastomere, for example a 16-32 cell mass (morula), or a cell derived from a porcine blastocyst, such as a totipotent cell from the inner cell mass of the blastocyst. The donor cell may be subject to conventional recombinant DNA manipulation where the DNA within the cell has been subject to recombinant DNA technology. For example, genes may be deleted, duplicated, activated or modified by gene additions, gene targeting, gene knock-outs, transgenesis with exogenous constructs which may or may not contain selectable markers may be accomplished by techniques such as microinjection, electroporation, viral-mediated transfection, lipofectin, calcium-phosphate precipitation (Lovell-Badge, “Introduction of DNA into embryonic stem cells” in: Teratocarcinomers and Embryonic Stem Cells: A Practical Approach, IRL Press, Oxford, E. J. Robertson, ed. pp 153-182, 1987; Molecular Cloning: A Laboratory Manual, Volume 2 & 3, Cold Spring Harbor Laboratory Press, Cold Spring Harbor, Sambrook, Fritsch and Maniatis Ed. pp 15.3-15.50, 16.3-16.68, 1989).

The resulting nuclear transferred cell following transfer of the nucleus of the porcine donor karyoplast into an enucleated porcine oocyte may be incubated in culture medium to allow one or more cell divisions to give a plurality of porcine embryonic cells. Porcine embryonic cells as referred to herein have the capacity, on implantation into a pregnancy competent porcine uterus, to develop into a porcine foetus. Porcine embryonic cells may contain, for example, 1, 2, 4, 8, 16 or 32 cells, or more. Cell division is a relatively rapid event and can be monitored by microscopic analysis. The porcine embryonic cells may be used directly for the production of cloned pigs, or alternatively may be conveniently stored, such as by being frozen in liquid nitrogen for subsequent use.

The nuclear transferred cell may be incubated to form a 2 to 32 cell mass, such as a 2 to 16 cell mass, whereafter the cell mass is synchronized at the G1 or G0 state as mentioned above. An nuclear transferred karyoplast may be isolated from the cell mass, and transferred into a second enucleated oocyte at the Metaphase II stage of development to give a second nuclear transferred cell, which may be cultured in vivo to allow one or more cell divisions to give porcine embryonic cells.

A single nuclear transferred porcine embryonic cell or plurality of cells produced according to this invention may be treated with an agent, such as cytochalasin B, so as to prevent cell division, but not nuclear division, whereafter multiple karyoplasts may be removed therefrom and used for subsequent nuclear transfer according to methods described herein (which may be regarded as serial nuclear transfer). Porcine embryonic cells as referred to herein include those treated with an agent such as cytochalasin B, or other agents.

In accordance with another aspect of this invention a nuclear transferred porcine embryonic cell or plurality of cells is treated with an agent which prevents cell division but not nuclear division, such that a karyoplast isolated therefrom is derived from a cell having multiple nuclei.

In another aspect of this invention there is provided a process for the clonal generation of pigs which process comprises providing a porcine oocyte at the Metaphase II stage of development from which the nucleus is removed, transferring a porcine donor karyoplast at the G1 state into the oocyte to give an NT cell, culturing the NT cell in vivo to allow successive cell division to give nuclear transferred porcine embryonic cells, and thereafter transferring a plurality of porcine embryonic cells so produced into a pregnancy competent uterus of a female pig which at conclusion of the pregnancy term gives rise to a plurality of genetically identical off-spring.

The clonal generation of pigs generally involves introducing into a pregnancy competent uterine environment of a female pig a plurality of embryonic cells as herein described. For example, from 5 to 50 embryonic cells may be introduced into the uterine environment according to standard procedures as used in the animal husbandry field or embryo development in gestational animals. The blastocysts may be inserted into the uterus using an appropriate device, such as a catheter or alternatively may be introduced into a fallopian tube for passage into the uterus. Non surgical procedures may also be used. The recipient female animal may be primed with the embryonic cells at or about the time of ovulation which may occur naturally, or as a result of induction according to established procedures such as by administration of appropriate hormonal regimes known in the art.

According to a further aspect there are provided genetically identical pigs when prepared according to the above process.

In another aspect this invention relates to progeny of pigs produced according to this invention (which may be referred to as nuclear transfer pigs (or NT pigs)). Progeny result from crossing an NT pig with another pig to give offspring piglets, that is progeny. The other pig may be an NT pig or any other pig (for example selected for a particular trait). A progeny animal contains a part of the genetic complement of the original porcine donor karyoplast, which can be conveniently detected, for example, by DNA markers.

According to another aspect of this invention there is provided a cloned pig produced from a nuclear transferred (NT) porcine embryonic cell. The present invention as described herein provides for implantation competent nuclear transferred cells that give rise to cloned pigs. In this regard the progeny or cloned pigs contain the identical DNA to that of the karyoplast used in their production as described herein. Accordingly animals of significant agricultural fitness may be produced expressing desired beneficial traits such as low fat meat, rapid growth, resistance to disease or suitability of organs for transplantation.

In a further aspect this invention relates to the use of cloned pigs as herein described in agriculture, for organ production, or oocyte and embryo production. The capacity to clonally manipulate pigs means that desirable characteristics can be directly exploited in the aforementioned areas. Thus, in agriculture, low fat meat can be produced by usage of a donor karyoplast expressing such a characteristic or induced to express such a characteristic by means of genetic manipulation, such as homologous recombination. By such an approach, the cloned pigs can be used in general for highly efficient and desirable agricultural pursuits, for organ production for use in human transplants (for example, where antigens have been removed, masked or attenuated by means such as genetic manipulation, for example homologous recombination), or for oocyte and embryo production.

The present invention will now be described with reference to the following non-limiting examples.

### EXAMPLE 1

Collection of Oocytes from Sows

Pregnant crossbred Large White X Landrace sows were aborted by intramuscular (IM) injection of 1 mg prostaglandin F2 analog (Cloprostenol; Estrumate, Pitman-Moore, NSW, Australia) between twenty five and forty days after mating followed by a second injection of 0.5 mg Cloprostenol twenty four hours later. One thousand international units of eCG (Pregnecol, Heriot AgVet, Vic, Australia) was administered (IM) at the same time as the second injection of Cloprostenol. Ovulation was induced by an IM injection of 500 iu hCG (Chorulon, Intervet, NSW, Australia) administered approximately seventy two hours after hCG. Oocytes were collected by surgically flushing oviducts forty eight to fifty two hours after hCG injection.

Culture of Ova

In vitro culture of oocytes, embryos and nuclear transfer embryos was conducted in 25 μl droplets of Whitten's medium (Whitten W K, 1971, in G Raspe, ed Advances in the Biosciences, Pergamon Press: Oxford, pp 129-141) supplemented with 15 mg/ml bovine serum albumin (BSA) under paraffin oil in a plastic petrie dish under an atmosphere of 5% CO₂, 5% O₂ and 90% N₂ in humidified air at 38-6° C.

### EXAMPLE 2

Enucleation of Oocytes

Oocytes were enucleated by aspirating the first polar body and adjacent cytoplasm (approximately 20% of cytoplasm) using a bevelled pipette (40 μm in diameter) in PB1+10% Fetal Calf Serum containing 7.5 μg/ml Cytochalasin B+5 μg/ml Hoechst 33342 (Sigma). Enucleation was confirmed by fluorescent staining of the aspirated portion of cytoplasm. Enucleated oocytes were cultured in Whitten's medium (WM) in a 5% CO₂ incubator until reconstruction of karyoplasts.

Reaggregation, Fusion and Activation of NT Cells

An individual karyoplast was inserted into the perivitelline space of each enucleated oocyte. The karyoplast-oocyte complexes were cultured in WM medium until activation and fusion. Fusion and activation of the karyoplast-oocyte complexes was induced using a BTX Electro Cell Manipulator ECM 2001. The complexes were first washed in fusion medium containing 0.3M Mannitol/100 μM CaCl₂ μM MgSO₄/0.01% polyvinylalcohol and then placed between two wire electrodes (1 mm apart) of the fusion chamber (450-10WG, BTX, CA) with 0.1 ml of fusion medium. Activation and membrane fusion may be induced by two sets of DC pulses (for example 150 v/mm, 60 μs) spaced from 5 seconds to one hour apart, preferably 30 minutes apart with a pre- and post-pulse alternating current (AC) field of 45 v, 1 MHz for 5 seconds each. Each set of DC pulses may comprise 1 or 2 closely spaced pulses. Where DC pulses are employed (a couplet) the pulses may be spaced from 1 to 20 seconds. NT embryos were placed in culture medium with or without cytochalasin B (CB) 7.5 μg/ml for 1-3 hours immediately following activation. Whilst not essential to the invention CB is used to prevent expulsion of chromosomes and aneuploidy following activation.

TABLE 1
Metaphase arrest induced in porcine blastomere nuclei following
treatment with nocodazole (NZ) dose × duration

| Duration of exposure | NZ concentration | Blastomeres at M | |
|---|---|---|---|
| 4 h | 1 μg/ml | 14/58 | (24%) |
| 7 h | 1 μg/ml | 54/133 | (41%) |
| 15 h | 1 μg/ml | 257/267 | (96%) |
| 15 h | 0.5 μg/ml | 101/133 | (76%) |
| Control | — | 15/291 | (5%) |

Results obtained are shown in the following tables:

TABLE 2
In vitro development of porcine morulae following NZ treatment

| Repeats | Duration | Dose | Development to blastocyst |
|---|---|---|---|
| 4 | 15 h | 1 μg/ml | 17/30 (57%) |
| 3 | 15 h | 0.5 μg/ml | 14/20 (70%) |
| 3 | 15 h | Control | 15/20 (75%) |

TABLE 3
Nuclear transfer results using karyoplasts at three different
stages of the cell-cycle

| Karyoplast | Cytoplast | No. | | 2-cell | 4-cell | Morula | Blastocysts | Cell No. of |
|---|---|---|---|---|---|---|---|---|
| stage | stage | reps. | n | (%) | (%) | (%) | (%) | blastocysts |
| S-phase | S-phase | 7× | 159 | 85^{a} | 35^{b} | 6^{b} | 6^{a} | 32.5 ± 4.0 |
| | | | | (53) | (22) | (10) | (4) | |
| Metaphase | M II | 3× | 53 | 29 | 10^{b} | 2^{b} | 0^{b} | — |
| | | | | (55) | (19) | (4) | (0) | |
| Gl | M II | 4× | 42 | 30^{b} | 20^{c} | 12^{c} | 9^{c} | 26.3 ± 3.4 |
| | | | | (71) | (48) | (29) | (21) | |

Legend:
Within each column, numbers with different superscripts are significantly different (P < 0.05).
S phase was achieved by oocyte activation; Metaphase was achieved by treatment with nocodazole;
S phase was achieved by treatment with nocodazole and aphidocolin (Verma et al (1999). Therio 51, 215).

### EXAMPLE 3

Development of Nuclear Transfer Embryos Derived from Differentiated Cells at G1

Foetal fibroblasts were isolated from d 25 porcine embryos (although embryos of other ages are also usable).

About 60% of cells in isolated unsynchronized foetal fibroblast cell populations are at the G1 phase of the cell cycle.

Foetal fibroblasts synchronised at G1 were prepared by isoleucine deprivation in in vitro culture. Cells were incubated in isoleucine-free RPM1 with 10%, foetal bovine serum for 2 d.

Unsynchronized and synchronized cells were used as karyoplasts to prepare nuclear transfer embryos, and morulalblastocyst development was determined. Results are shown in Table.

TABLE 4

| | | | | | | Morula/ |
|---|---|---|---|---|---|---|
| | reps | n | fused | 2 cell | 4 cell | blastocyst |
| Unsynchronised | 4× | 84 | 65 | 51 | 24 | 2(3) |
| | | | (79) | (40) | | |
| G1 synchronised | 4× | 122 | 91 | 62 | 29 | 6(7) |
| | | | (68) | (32) | | |

Legend:
Numbers in brackets are percentage of oocytes fused.
Results show that porcine nuclear transfer embryos can be derived from differentiated karyoplast at G1.

### EXAMPLE 4

Embryo Transfer of Nuclear Transfer Embryo

Pregnant crossbred Large White X Landrace sows are aborted by intramuscular (TM) injection of 1 mg prostaglandin F2 analog (Cloprostenol; Estrumate, Pitman-Moore, NSW, Australia) between twenty five and forty days after mating followed by a second injection of 0.5 mg Cloprostenol twenty four hours later. Five hundred international units of eCG (Pregnecol, Heriot AgVet, Vic, Australia) is administered (IM) at the same time as the second injection of Cloprostenol. Ovulation is induced by an IM injection of 500 iu hCG (Chorulon, Intervet, NSW, Australia) administered approximately seventy two hours after eCG. Twenty-five to thirty, 4-cell embryos surgically transferred to the oviduct of a sow seventy two hours after the hCG injection result in a litter of 5 to 8 piglets following a successful pregnancy.

### EXAMPLE 5

Production of NT Embryos

Oocytes were collected from superovulated Large White x Landrace donor pigs 48-52 h post hCG, and denuded of cumulus by pipetting and hylauronidase treatment. Oocytes were enucleated by removal of the first polar body and adjacent cytoplasm, and activated and fused to foetal fibroblasts simultaneously at 54-56 h post hCG using two sets of DC pulses (1.5 kV/cm, 60 μs×2) given 30 minutes apart in 0.3M mannitol solution containing 0.1 mM CaCl₂, 0.1 mM MgSO₄ and 0.01% PVA. NT embryos were placed in culture medium with or without cytochalasin B (CB) 7.5 ±g/ml for 1-3 hours immediately following activation. CB is used to prevent expulsion of chromosomes and aneuploidy following activation. Fibroblasts were obtained from day 25 fetuses and cultured in DMEM plus 10% FBS. Cells at passage 3 to 5 were made quiescent (that is, in the G0 phase) by culture for 5 days at 0.5% FBS. For example, early passage foetal fibroblasts were plated at a density of 5×10⁴/cm² in DMEM+10% foetal bovine serum. After 48 hours the medium was changed to DMEM+0.5% % foetal bovine serun. 5 days later the cells were harvested by tripsin digestion and resuspended in DMEM+10% foetal bovine serun. NT embryos were cultured in 25 μl droplets of NCSU23 (Petters and Wells (1993), J. Reprod. Fert. Suppl. 48, 61-73) with 0.4% bovine serum albumin (BSA) under paraffin oil in a plastic petri dish under an atmosphere of 5% CO₂, 5% O₂, 90% N₂ at 38.6° C. 10% foetal bovine serum was added.

The in vitro development of NT embryos using this procedure is shown in Table 4.

TABLE 5
Development of nuclear transfer embryos
constructed with porcine fetal fibroblasts

| | | | development | | |
|---|---|---|---|---|---|
| | no. successfully | | to (%) | | |
| no. oocytes | fused | 2 cell | 4 cell | morula | blastocyst |
| 127 | 103 | 58(56) | 27(26) | 7(7) | 3(3) |

data is the sum of 5 replicates
numbers in brackets are percentage of embryos which successfully fused.

Fusion and activation rates obtained using porcine fetal fibroblasts were similar to those reported for sheep and cattle previously (loc. cit.). However, development to the blastocyst stage was lower suggesting that there is a difference between the pig and these species in the ease with which fetal fibroblast nuclei can be reprogrammed using current nuclear transfer methods.

Transfer of NT Embryos

Embryos produced using the above method were transferred to recipient animals to allow them to develop to term. The protocol used is described below.

Because the recipient oocyte is damaged during the nuclear transfer process, the majority embryos were encapsulated in agar (or agarose) to maintain their integrity and prevent immunological attack.

Because in vitro culture conditions do not mimic those in vivo, NT embryos were transferred to the ligated oviduct of a mated recipient the day after reconstruction to maximize development.

Transferred embryos were collected 3 to 4 days later and morula and blastocyst embryos transferred to the uterus of a mated or unmated second recipient. The type of recipient used depended on the number of NT and in vivo derived embryos recovered from the first recipient. When this number was low (<10), embryos were transferred to a mated recipient to maximize the potential for their development.

The number of NT embryos transferred, the type of second recipient used and pregnancy outcome is shown in Table 5.

TABLE 6
Transfer of NT Embryos

| | No. NT | No. NT + in vivo | | |
|---|---|---|---|---|
| | embryos | derived embryos | Transferred | |
| | transferred | recovered/transferred | to unmated | Pregnancy |
| | to temporary | from temporary | or mated | status of |
| Date | recipient | recipient | 2nd recipient | recipient |
| 2/10 | 40 | 8M + 2BNT + 10C | unmated | 9 piglets |
| | | | | born |
| 16/10 | 35 | 1M + 1BNT + 0C | mated | returned |
| 23/10 | 40 | 4MNT + 12C | unmated | 4 piglets |
| | | | | born |
| 30/10 | 31 | 1BNT + 1C | mated | 6 piglets |
| | | | | born |
| 6/11 | 39 | 1M + 2BNT + 10C | mated/one | pregnant |
| | | | side flushed | |
| 27/11 | 40 | 1M + 1BNT + 9NT + 7C | mated | pregnant |
| 1/12 | 67 | 4M + 4NT + 18C | unmated | returned |
| 4/12 | 42 | 3MNT + 8C | mated/one | pregnant |
| | | | side flushed | |
| 8/12 | 46 | 4MNT + 3C | mated/one | returned |
| | | | side flushed | |
| 11/12 | 38 | 5MNT + 1BNT + 7C | unmated | returned |
| 15/12 | 36 | 3MNT + 13C | unmated | pregnant |
| 18/12 | 42 | 4NT + 14C | unmated | ♦ |

NT = 4-8 cell NT embryos
MNT = NT embryos at morula stage
BNT = NT embryos at blastocyst stage
C = carrier embryos not derived by NT
# pregnancy determined using real time ultrasound
♦ under analysis

Pregnancy may be terminated at any stage to provide easy analysis of the genotype of implanted embryos. Identification of implanted embryos with the genotype of karyoplasts used in nuclear transfer verified implantation capacity of nuclear transfer embryos.

Throughout this specification, unless the context requires otherwise, the word “comprise”, or variations such as “comprises” or “comprising” or the term “include” or variations thereof, will be understood to imply the inclusion of a stated element or integer or group of elements or integers but not the exclusion of any other element or integer or group of elements or integers. In this regard, in construing the claim scope, an embodiment where one or more features is added to any of claims is to be regarded as within the scope of the invention given that the essential features of the invention as claimed are included in such an embodiment.

1. A process for the production of nuclear transferred porcine embryonic cells which includes providing a porcine oocyte at the Metaphase II stage of development from which the nucleus is removed, transferring a porcine karyoplast at the G0 or G1 state into the oocyte to give a nuclear transferred porcine embryonic cell and optionally culturing the nuclear transferred cell in vitro to allow one or more cell divisions to give a plurality of nuclear transferred porcine embryonic cells.

2. A process according to claim 1wherein the nuclear transferred porcine embryonic cell or plurality of cells, such as a 2 to 32 cell mass, is synchronized at the G0 or G1 state, isolating a nuclear transferred karyoplast therefrom, and transferring said karyoplast into a second enucleated oocyte at the Metaphase II stage of development or to an enucleated zygote, or later stage embryo or embryonic cell to give a second nuclear transferred cell, which may be cultured in vitro to allow one or more cell divisions to give a plurality of nuclear transferred porcine embryonic cells.

3. A process according to claim 2 wherein the nuclear transferred porcine embryonic cell or plurality of cells is treated with an agent which prevents cell division but not nuclear division, such that a karyoplast isolated therefrom is derived from a cell possessing multiple nuclei.

4. A process for the production of porcine embryonic cells wherein the method of claim 3 is repeated a plurality of times.

5. A process for the clonal generation or propagation of pigs which process includes providing a porcine oocyte at the Metaphase II stage of development from which the nucleus is removed, transferring a porcine donor karyoplast at the G0 or G1 state into the oocyte to give a nuclear transferred porcine embryonic cell, and thereafter culturing the nuclear transferred cell in vitro to allow one or more cell divisions to give a plurality of nuclear transferred porcine embryonic cells, and thereafter transferring a plurality of porcine embryonic cells so produced into a pregnancy competent uterus of a female pig which at conclusion of the pregnancy term gives rise to one or more genetically identical off-spring.

6. A process according to claim 1 wherein a karyoplast is synchronized at the G1 state by use of DNA synthesis inhibitor and/or a microtubule inhibitor and/or use of means which do not involve serum starvation of cells.

7. A process according to claim 1 wherein a karyoplast is synchronized at the G0 state by nutrient deprivation or chemical treatment.

8. A process according to claim 1 in which the karyoplast is genetically altered or modified.

9. A process according to claim 6 where microtubule inhibition is achieved by the application of nocodazole.

10. A process according to claim 1 wherein karyoplast synchronization at G1 is achieved by the application of aphidicolin.

11. A process according to claim 1 wherein the porcine karyoplast at the G0/G1 state is fused and activated in an enucleated porcine oocyte at the Metaphase II stage of development by application of multiple electrical pulses spaced in their order of application, or by other means of generating multiple transient increases in intracellular Ca levels.

12. A process according to claim 11 wherein from 1 to 6 pulses are delivered at an interval between each pulse of from one minute to sixty minutes.

13. A process according to claim 12 wherein pulses are applied at a thirty minute interval.

14. A method according to claim 11 wherein each pulse is a set of pulses of 2 to 4 pulses, spaced from each other by 1 to 20 seconds.

15. Porcine embryonic cells or cloned pigs when produced according to a process comprising or including a process as defined in any preceding claim.

16. Progeny of a pig according to claim 15.

17. A cloned pig produced from a nuclear transferred porcine embryonic cell.

18. Use of cloned pigs in agriculture, for organ production, or oocyte and embryo production.
